(19) United States
(12) Patent Application Publication
(10) Pub. No.: US 2020/0292539 A1
(43) Pub. Date: Sep. 17, 2020

(54) RESULT DETERMINATION IN AN IMMUNOASSAY BY MEASURING KINETIC SLOPES

(71) Applicant: Quidel Corporation, San Diego, CA (US)

(72) Inventors: Peter Yan-Guo REN, San Diego, CA (US); Stewart HOELSCHER, San Diego, CA (US); Cristian ALBERTO, San Diego, CA (US); Stephanie PINEDO, San Diego, CA (US); Jason MCCLURE, San Diego, CA (US); Dipesh Jaiswal, San Diego, CA (US)

(21) Appl. No.: 16/817,010

(22) Filed: Mar. 12, 2020

Related U.S. Application Data
(60) Provisional application No. 62/818,403, filed on Mar. 14, 2019.

(51) Int. Cl. G01N 33/543; G01N 21/27; G01N 21/84

(52) U.S. Cl. CPC G01N 33/54386; G01N 2021/6439; G01N 21/8483; G01N 21/274

(57) ABSTRACT

A system including a sample receptacle configured to receive a test sample and a test strip coupled to the sample receptacle is provided. The test strip configured to generate a signal based on a concentration of a target analyte in the test sample. The system also includes a detector to generate a transduced signal based on the signal and a computer to receive a transduced signal. The computer further determines the concentration of the target analyte in the test sample. For this, the computer retrieves the transduced signal from the detector at multiple time points to determine a signal rate based on a signal value for the time points, and to determine the concentration of the target analyte based on the signal rate and a model. A method and a non-transitory, computer-readable medium storing instructions to use the above system are also provided.

### CROSS-REFERENCE TO RELATED APPLICATIONS

The present application claims priority under 35 U.S.C § 119(e) to U.S. Provisional Patent Application No. 62/818,403, filed on Mar. 14, 2019, the contents of which are hereby incorporated by reference in their entirety, for all purposes.

### TECHNICAL FIELD

The technology described herein generally relates to assay measurements, and more particularly relates to quantitative determination of endpoints in assay measurements.

### BACKGROUND

Immunoassays are widely used to detect the presence and/or concentration of an analyte in a test sample, such as an antibody. Most such assays rely on the specific binding between the antibody and an antigen. In an endpoint assay, just one data point is collected—say at 5 minutes after the assay starts, at which point the laboratory staff collects the data or develops the strip. The outcome is qualitatively a positive or negative reading. In this type of assay, it is assumed that the resulting concentration of the molecule or complex to be measured is directly proportional to the reaction time. Thus, there is no rate of change of production to be recognized or titrated.

While much attention has been paid to the nature of the antibody/antigen pair and technical methods of improving binding efficiency, surprisingly the endpoint (e.g., whether a binding event has occurred) is typically based on a single measurement. There is thus the real possibility of a simple error in the endpoint determination and little possibility of determining any qualitative aspects of binding with just a single measurement. For example, errors may occur when a sample presents a wide range of disparate reaction times across a population of positive samples.

Furthermore, when the endpoint is determined based on a single parameter (e.g., a particular time-point or a particular analyte concentration), variations in endpoint determination from sample to sample may result from fluctuations in ambient conditions. Accordingly, there is a need for a more reliable method of endpoint determination in an immunoassay.

The discussion of the background herein is included to explain the context of the technology. This is not to be taken as an admission that any of the material referred to was published, known, or part of the common general knowledge as at the priority date of any of the claims found appended hereto.

Throughout the description and claims of the instant application the word “comprise” and variations thereof, such as “comprising” and “comprises,” is not intended to exclude other additives, components, integers, or steps.

### SUMMARY

The instant disclosure addresses a method of determining the cut-off for concentrations of analyte in qualitative or quantitative immunoassays, the method based on calculating the kinetic slope, or rate, of the immunoassay over time. Thus, an endpoint assay is effectively converted into a kinetic assay by virtue of multiple readings being made even though the rate is constant throughout the duration of the assay.

In a first embodiment, a method of determining an endpoint in an assay includes measuring a sample signal from a test sample at a plurality of time points in an assay formed on a test strip. The sample signal correlates with a concentration of a target analyte in the test sample. The method also includes determining a rate value of the sample signal over a duration of the assay based on the sample signal at the plurality of time points and providing a result of the assay according to the rate value and a pre-selected threshold.

In a second embodiment, a system includes a sample receptacle configured to receive a test sample and a test strip coupled to the sample receptacle, the test strip configured to generate a signal based on a concentration of a target analyte in the test sample. The system also includes a detector configured to generate a transduced signal based on the signal and a computer configured to receive the transduced signal. The computer further includes a memory storing instructions and a processor configured to execute the instructions to determine the concentration of the target analyte in the test sample. The instructions include commands to retrieve the transduced signal from the detector at multiple time points, to determine a signal rate based on a signal value for at least two of the time points, and to determine the concentration of the target analyte based on the signal rate and a model.

In yet another embodiment, a non-transitory, computer-readable medium includes instructions which, when executed by a processor, cause a computer to perform a method. The method includes measuring a sample signal at a plurality of time points in an assay formed on a test strip. The sample signal correlates with a concentration of a target analyte in a test sample generating the sample signal. The method also includes determining a rate value of the sample signal over a duration of the assay based on the sample signal at the plurality of time points, and providing a result of the assay according to the rate value and a pre-selected threshold.

### BRIEF DESCRIPTION OF THE DRAWINGS

FIG. 1 illustrates a system for analyzing a test sample in an assay, according to some embodiments.

FIG. 2 illustrates an assay including multiple test channels in a sample cartridge for parallel measurement, according to some embodiments.

FIG. 3 illustrates an area of interest in a test sample configured for a lateral diffusion immunoassay, according to some embodiments.

FIG. 4 is a chart illustration of signal vs. time curves for positive and negative samples in an assay, according to some embodiments. In some embodiments, the slope is constant and the curves are straight lines indicating that the signal linearly increases with time, with no inflection point.

FIG. 5 is a chart illustrating the slope of the curves in FIG. 4 as a function of analyte concentration. For quantitative assays, the kinetic slopes are linear against analyte concentrations.

FIG. 6 is a chart illustrating the slope of the curves in FIG. 4 as a function of analyte concentration in a Michaelis-Menten Kinetics, according to some embodiments.

FIG. 7 is a chart illustrating the slope of the curves in FIG. 4 as a function of analyte concentration according to a 4-PL model, according to some embodiments.

FIG. 8 is a flow chart illustrating steps in a method for determining an endpoint in an assay, according to some embodiments.

FIG. 9 is a flow chart illustrating steps in a method for determining an endpoint in an assay with a remote server, according to some embodiments.

FIG. 10 is a block diagram illustrating a system to implement at least partially the methods as disclosed herein, according to some embodiments.

Like reference symbols in the various drawings indicate like elements.

### DETAILED DESCRIPTION

The methods described herein are applicable to any type of endpoint assay, in particular an immunoassay such as enzyme-linked immunosorbent assays (ELISA) or a lateral flow assay, and assays based on a rapid diagnostic strip, and are irrespective of the antibody/antigen pair. Other types of assays for which the methods can be applied include chemical assays, microbiological assays, and bioassays generally.

The methods described herein are applicable to many sorts of binding measurements and can be based on measurable signals from sources such as radioactive decay, fluorescence, or chemi-luminescence. Measurements from other techniques such as colorimetry, photometry, spectrophotometry, and chromatography can also be used.

The methods described herein can be applied to determining a qualitative or a quantitative result to an assay for a test sample, in-situ or remotely.

In the methods described herein, measurements at multiple time points are made during the course of an immunoassay reaction in order to establish time-dependent signal profiles. From such measurements, the slope (sometimes referred to as the rate) of the signal vs. time can be calculated and compared to a cut-off or threshold slope for that assay.

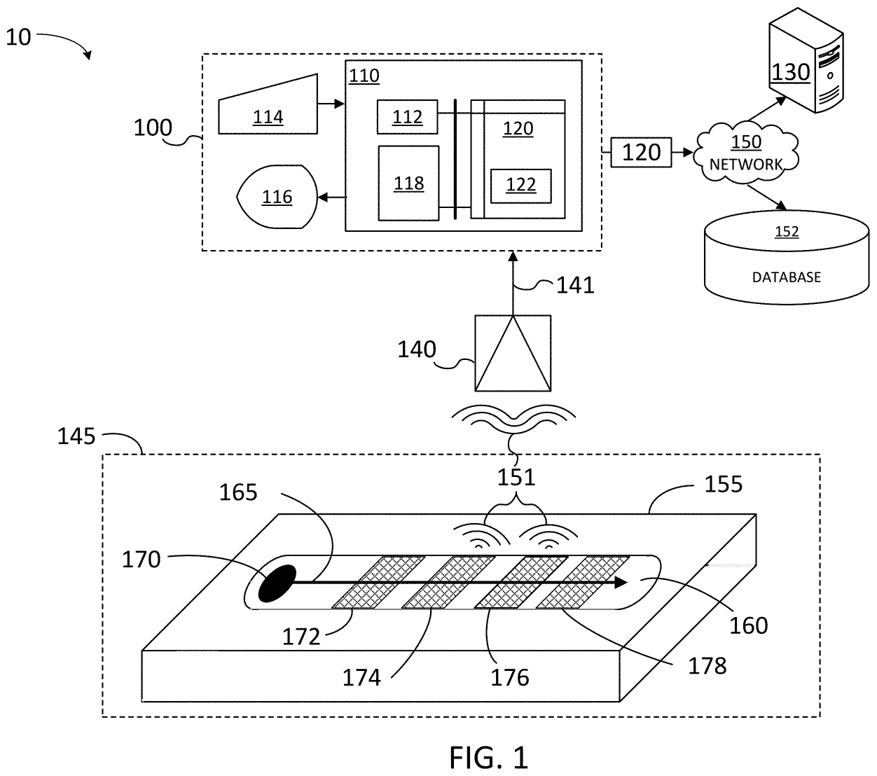

FIG. 1 illustrates a system 10 for analyzing a test sample in an assay, according to some embodiments. The system includes a test kit 145, a detector 140, and an analysis device 100. Test kit 145 includes a sample cartridge 155 with a test strip 160 configured to receive a sample in a sample receptacle 170 and generate a signal 151 from at least a test band 174 and a control band 176. Signal 151 is collected by detector 140. Detector 140 converts signal 151 into a transduced signal 141. Analysis device 100 receives transduced signal 141 and generates a result 120. In some embodiments, test kit 145 and detector 140 may be assembled in the same encasing. In some embodiments, detector 140 may be part of analysis device 100. Further, in some embodiments, analysis device 100 includes one or more computers communicably coupled with each other, to share and store data and information associated with test kit 145, detector 140, and the sample (e.g., the origin of the sample: person, animal, plant, location, venue, time of collection, and the like).

Analysis device 100 includes a computer station having a processor 112, a memory 120, and a communications module 118. Memory 120 stores data associated with sample test provided to sample receptacle 170 (e.g., results 120). Memory 120 also stores instructions which, when executed by processor 112, cause analysis device 100 to perform at least partially some of the steps in methods consistent with the present disclosure. In some embodiments, the instructions are stored in the form of an application (software) 122 installed in the computer station. Further, in some embodiments application 122 may be hosted by a remote server 130 via communications module 118. Communications module 118 may include wireless radios and network communication devices and protocols. Communications module 118 is configured to interface with detector 140 and also to communicate with one or more stations or computers locally or remotely, which may be part of analysis device 100. In some embodiments, analysis device 100 is configured to provide result 120 to remote server 130 for storage or further analysis (e.g., during an endemic, epidemic or pandemic event, an environmental emergency—food and drug administration—and the like). Analysis device 100 may also include an input device 114 and an output device 116, for a user interface. Input device 114 may include a mouse, a keyboard, a touchscreen display, a microphone, or a webcam to receive commands from a user. Likewise, output device 116 may include a display (e.g., a touch screen device), a printer, an alarm, or a warning light, to indicate to the result of the sample test to the user.

In some embodiments, analysis device 100 performs a partial data processing of transduced signal 141, and provides a partial result 120 and a partial transduced signal 140 to remote server 130 through network 150. Moreover, in some embodiments analysis device 100 transmits the entire transduced signal 140 to server 130, for data processing. Accordingly, in some embodiments analysis device 100 simply collects transduced data 140 and provides it to server 130. The data analysis may then be performed by one or more remote servers 130. In yet other embodiments, at least some of the analysis of transduced signal 140 may be carried out by any computer device (e.g., a smartphone, laptop, desktop and the like) or large parallel computer system communicatively coupled to network 150. In some embodiments the final results may be transmitted back to analysis device 100 or to a separate device (cell phone, laptop, desktop, and the like) via a secure connection through network 150. In some embodiments, analysis device 100 may also be communicatively coupled with a database 152 through network 150. Database 152 may include a server configured to store transduced data 141 and results 120 from one or more of analysis devices 100 in system 10.

In some embodiments, application 222 may include a multilinear regression algorithm or a nonlinear algorithm to process transducer signal 140. For example, and without limitation, in some embodiments application 222 may include an artificial intelligence or machine learning algorithm trained to process transducer signal 140 and provide results, according to embodiments disclosed herein. In some embodiments, the artificial intelligence or machine learning algorithms may include neural networks, convolutional neural networks, deep learning neural networks and the like. The non-linear algorithms may be trained on historical data from multiple analysis devices 100 over multiple samples from multiple patients or subject, stored in database 152, and accessible to server 130 or analysis device 100 through network 150.

The test kit includes a sample cartridge configured to receive a test sample in a sample receptacle, and a test strip. In some embodiments, the sample is a fluid or is dissolved in a fluid, and is fluidically coupled with the test strip. The test strip may include a fibrous or porous material that induces capillary diffusion of the fluid in a downstream direction, towards a test band and a control band, among other components in the test strip. In some embodiments, a label pad includes label complexes that dissolve in a test sample as it diffuses along the test strip. The label complexes are configured to emit a signal, and to attach to any target analyte present in the test sample. The test band may include fixation elements configured to capture the target analyte-label complex compound. The control band may be a blank portion of the diffusing matrix (e.g., tissue, paper, strip, gel and the like) configured to provide a background signal. In some embodiments, an absorbent pad is configured to absorb the remaining test sample (including some residual target analyte-label complex compounds).

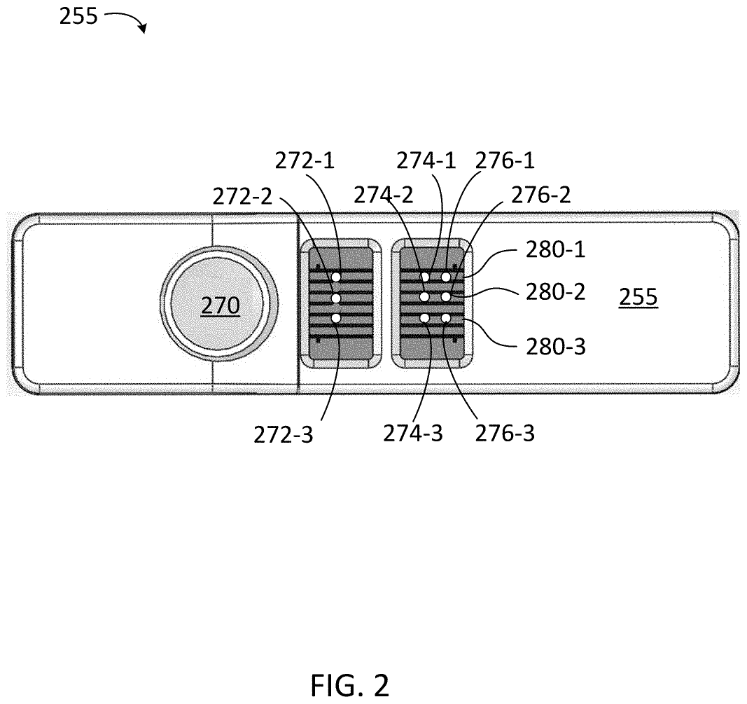

FIG. 2 illustrates an assay including multiple test channels 280-1, 280-2 and 280-3 (hereinafter, collectively referred to as “test channels 280”) in a sample cartridge 255 for parallel measurement, according to some embodiments. In some embodiments, each one of test channels 280 may include a replicate test with separate test dots 274-1, 274-2, and 274-3 (hereinafter, collectively referred to as “test dots 274,” e.g., test band 174) respectively. In some embodiments, test dots 274 may include binding members for the same analyte of interest. In some embodiments, test dots 274 may include binding members for different analytes of interest (e.g., for multi-component assays). Test channels 280 also include separate control dots 276-1, 276-2 and 276-3 (hereinafter, collectively referred to as “control dots 276,” e.g., control band 176), respectively. In some embodiments, test dots 274 and control dots 276 may each be associated with a specific fluorescent emission color (or wavelength), to distinguish from one another. Accordingly, in some embodiments each test channel 280 may be associated with a specific fluorescent emission color (‘red’, ‘green’, ‘blue’ and the like). In some embodiments, one of test channels 280 may be associated to more than one fluorescent emission color.

In some embodiments the result from each test channel 280 may be analyzed independently, in kinetic mode. In some embodiments, the results from test channels 280 may be analyzed jointly. Accordingly, a final result may be obtained during data analysis or after each channel result is collected. The final result may include an average of all, or most of the results from test channels 280. In some embodiments, the results from each of test channels 280 may be embedded within the kinetic analysis to yield a single result or a joint analysis of all sub-results, leading to a more accurate result.

In some embodiments, control dots 276 may constitute a set of quantitative standards for sample cartridge 255. Accordingly, a signal from control dots 276 may be used in the analysis device to report either a qualitative result(s) (with a fixed end point) or a quantitative result.

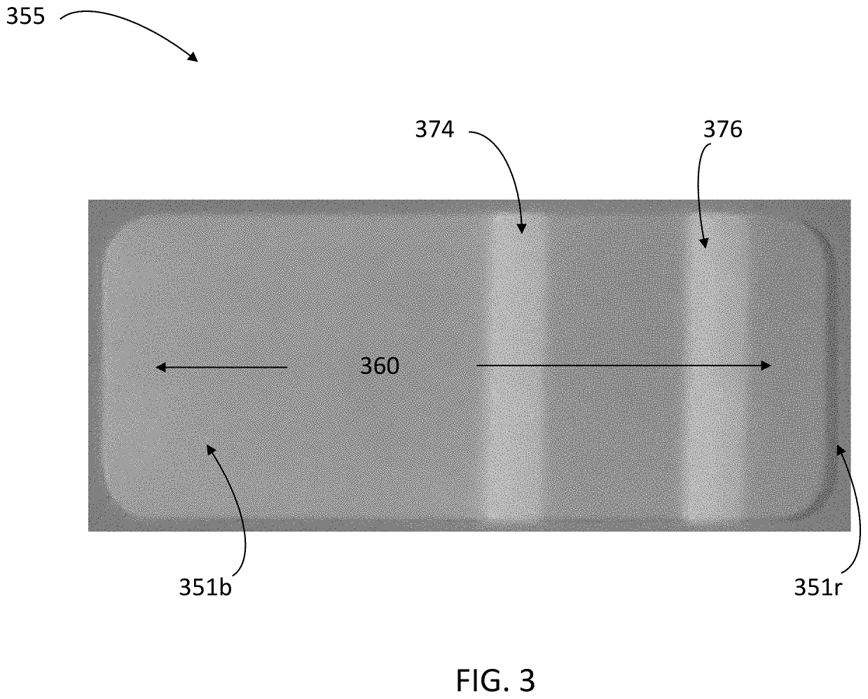

FIG. 3 illustrates an area of interest 360 in a sample cartridge 355 configured for a lateral diffusion immunoassay, according to some embodiments. Area of interest 360 includes a test band 374, a control band 376 and may further include a label pad, an absorbent pad, and a sample receptacle all included in sample cartridge 355 (cf. FIG. 1). For illustration purposes only, the assay in FIG. 3 is a fluorescence-based immunoassay, and thus test band 374 and control band 376 are highlighted by the fixation of fluorescence emitting labels. A residual background 351r may diffuse beyond control band 376, including a concentration level of fluorescent labels emitting residual background. In some embodiments a background signal 351b and residual background 351r may be used by application 122 in the computer station to filter the signal and provide an accurate result (cf. FIG. 1).

Sample cartridge 355, in one embodiment, is an immunoassay test strip enclosed in a housing or cartridge to ease its handling. In other embodiments, sample cartridge 355 is simply an immunoassay test strip, such as a dip stick. That is, an external housing is optional, and if present, need not be a cartridge or cassette housing but can be a flexible laminate, such as that disclosed in U.S. Patent Application Publication No. 2009/02263854 and shown in Design Patent No. D606664.

An immunoassay test strip, in one embodiment, comprises in sequence, a sample pad, a label pad, one or more lines or bands selected from test band 374, control band 376 and a reference band, and an absorbent pad. In some embodiments, a support member is present, and each or some of the sample pad, label pad, bands, and absorbent pad are disposed on the support member. Exemplary immunoassay test strips are described, for example, in U.S. Pat. Nos. 9,207,181; 9,989,466; and 10,168,329 and in U.S. Publication Nos. 2017/0059566 and 2018/0229232, each of which is incorporated by reference herein.

The immunoassay test strip may be configured uniquely for detection of a particular pathogen or analyte of species of interest. These include, but are not limited to, proteins, haptens, immunoglobulins, enzymes, hormones, polynucleotides, steroids, lipoproteins, drugs, bacterial antigens, and viral antigens. With regard to bacterial and viral antigens, more generally referred to in the art as infectious antigens, analytes of interest include Streptococcus, Influenza A, Influenza B, respiratory syncytial virus (RSV), hepatitis A, B, and/or C, pneumococcal, human metapneumovirus, and other infectious agents well-known to those in the art. In some embodiments, a test device is intended for detection of one or more of antigens associated with Lyme disease. In some embodiments, an immunoassay test strip is intended for use in the field of women's health. For example, test devices for detection of one or more of fetal-fibronectin, chlamydia, human chorionic gonadotropin (hCG), hyperglycosylated chorionic gonadotropin, human papillomavirus (HPV), and the like, are contemplated. In another embodiment, an immunoassay test strip for detection of vitamin D is designed for interaction with the apparatus and method of normalization described herein.

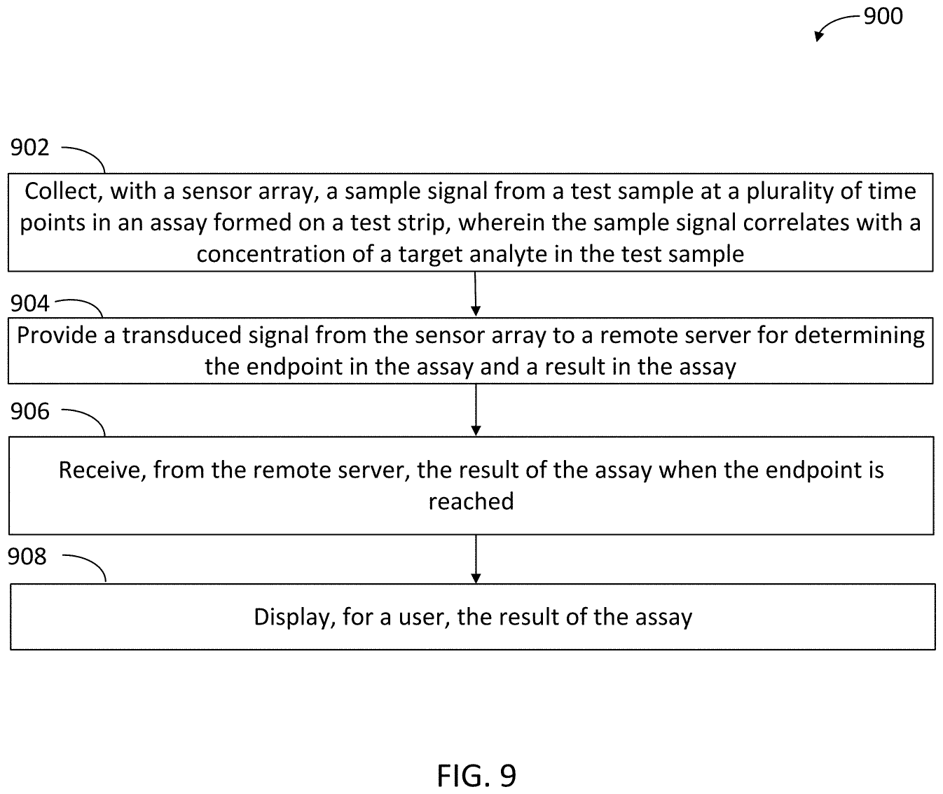

An exemplary immunoassay test strip including a sample receiving zone in fluid communication with a label zone may be as disclosed in FIGS. 9A and 9B of U.S. Pat. No. 9,207,181, or FIG. 3 of U.S. Pat. No. 9,989,466. A fluid sample placed on or in sample receiving zone flows by capillary action from sample receiving zone in a downstream direction. The label zone is in fluid communication with at least a test line or band and, optionally, a control line or band and/or a reference line or band. Typically, the label zone is downstream from the sample receiving zone, and the series of control and test bands are downstream from the label zone, and an optional absorbent pad is downstream from the portion of the test strip on which the bands are positioned.

The sample zone receives the sample suspected of containing an analyte of interest. The label zone, in some embodiments, contains two dried conjugates that include particles containing a label element. The label element includes a label that emits a signal in any of a number of selected emission processes: e.g., electromagnetic radiation, alpha particle radiation, positron radiation, beta radiation, and the like. In some embodiments, the electromagnetic radiation emission may include a fluorescence emission, Raman emission, and the like. Further, in some embodiments, the label may absorb a selected type of radiation, e.g., electromagnetic radiation as in microwave absorption, infrared (IR) absorption, visible absorption, or ultraviolet (UV) absorption. Further, in some embodiments, the label element may include multiple label elements selected from all or more of the above radiation emission and/or absorption described above.

Without loss of generality, and to illustrate the operation of the system at hand, in one embodiment the label element may include a fluorescent element. An exemplary fluorescent element is a lanthanide material, made by one, or a combination of the fifteen elements lanthanum, cerium, praseodymium, neodymium, promethium, samarium, europium, gadolinium, terbium, dysprosium, holmium, erbium, ytterbium, lutetium, and yttrium. In one embodiment, the lanthanide material is embedded in or on a particle, such as a polystyrene particle. The particles can be microparticles (particles less than about 1,000 micrometers in diameter, in some instances less than about 500 micrometers in diameter, in some instances less than 200, 150, or 100 micrometers in diameter) containing a luminescent or fluorescent lanthanide, wherein in some embodiments, the lanthanide is europium. In some embodiments, the lanthanide is a chelated europium. The microparticles, in some embodiments, have a core of a lanthanide material with a polymeric coating, such as an europium core with polystyrene coating. A binding partner for the analyte(s) of interest in the sample is/are attached to or associated with the outer surface of the microparticles. In some embodiments, the binding partner for the analyte(s) of interest is an antibody, a monoclonal antibody, or a polyclonal antibody. A skilled artisan will appreciate that other binding partners can be selected, and can include complexes such as a biotin and streptavidin complex. Upon entering the label zone, the liquid sample hydrates, suspends, and mobilizes the dried microparticle-antibody conjugates and carries the conjugates together with the sample downstream on the test strip to the control or reference and/or test bands disposed on the immunoassay test strip. If an analyte of interest is present in the sample, it will bind to its respective conjugate as the specimen and microparticles flow from the label zone.

As the sample and microparticle-antibody conjugates continue to flow downstream on the immunoassay test strip, if the analyte of interest is present in the sample, the fluorescent microparticle-antibody conjugate, which is now bound with antigen/analyte of interest, will bind to the specific binding member for the analyte of interest that is immobilized at the test band(s). In some embodiments, a single test band is present on the test strip. In some embodiments, at least two, or two or more test bands are present on the strip. By way of example, a test strip intended for detection and/or discrimination of influenza A and influenza B can include a first test band to detect influenza A and a second test band to detect influenza B. Microparticle-antibody conjugates comprised of microparticles coated with antibodies specific for influenza A and microparticles coated with antibodies specific for influenza B may be included in the label zone, and in some embodiments, downstream of the negative control band. A first test band for influenza A and a second test band for influenza B can be disposed downstream of the label zone. The first test band for influenza A comprises a monoclonal or polyclonal antibody to a determinant on the nucleoprotein of influenza A and the second test band for influenza B comprises a monoclonal or polyclonal antibody to a determinant on the nucleoprotein of influenza B. If an antigen is present in the sample, a typical immunoassay sandwich will form on the respective test band that matches the antigen in the sample.

The microparticle-antibody conjugates that do not bind to the negative control band or to a test band continue to flow by capillary action downstream, and the remaining sample encounters the reference band, in some embodiments proceeding into the absorbent pad.

The immunoassay test device is intended for receiving a wide variety of samples, including biological samples from human bodily fluids, including but not limited to, nasal secretions, nasopharyngeal secretions, saliva, mucous, urine, vaginal secretions, fecal samples, blood, etc.

The kit described herein, in some embodiments, is provided with a positive control swab or sample. In some embodiments, a negative control swab or sample is provided. For assays requiring an external positive and/or negative control, the user may be prompted to insert or apply a positive or negative control sample or swab.

An immunoassay band emits fluorescence light primarily from fluorophores bound to the target analyte, as they are fixed on the substrate by adherence to the immuno-proteins in the immunoassay strip (e.g., adsorption, chemi-sorption, immune-ligand, and the like). Accordingly, the presence of a red emission within the boundaries of the band is mostly attributable to the presence of the target analyte (e.g., presence of pathogenic antigens, and the like). However, the amount of red signal within the boundaries of the immunoassay band may include some background. To better assess the background signal (e.g., not originated by target analytes bound to the antibodies on the band), some sample cartridges may include a blank control area.

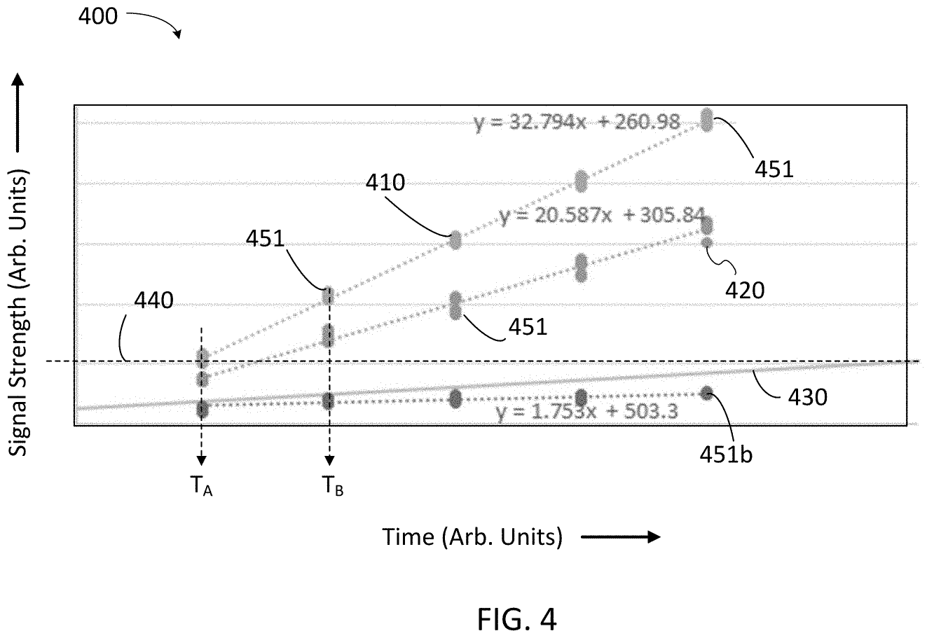

FIG. 4 is a chart 400 illustrating of signal vs. time curves for positive and negative samples in an assay, according to some embodiments. In some embodiments, the slope is constant and the curves are straight lines indicating that the signal linearly increases with time, with no inflection point. The abscissae includes time (X-axis, arbitrary units) and the ordinates include signal strength values (Y-axis, arbitrary units). The measurement values in chart 400 correspond to different assays running over three different test samples. For example, in some embodiments the data points in each of the data bunches 451 in chart 400 may correspond to different test channels in a single sample cartridge (e.g., test channels 280 in sample cartridge 255). In some embodiments, the data points in each of data bunches 451 may correspond to different sample cartridges. Accordingly, each data point in data bunches 451 may be separately analyzed in kinetic mode or jointly analyzed such that during data analysis or after each sub-result is analyzed the final result comes from an average of all sub-results or is embedded with the analysis to yield a single result or a joint analysis of all sub-results together leading to a more accurate result.

As it is seen, the slope (or rate) of the signal varies consistently depending on the concentration level of the target analyte in the test sample. A curve 410 with high-level target analyte shows a steeper slope relative to a curve 420 with a mid-level target analyte. Without loss of generality, the assays shown are fluorescence emission immunoassays configured to fix fluorescent labels to a pathogen “organism” (e.g., a bacterium or virus, the target analyte). For illustrative purposes only, and without loss of generality, the target analyte in chart 400 is Strep A, measured as organisms per milliliter (org/mL). The test strip in the assay includes a test band having antibody conjugates configured to fix the organisms to the substrate, thereby enhancing the fluorescence emission across the test band, compared to the rest of the test strip. Accordingly, the signal strength may indicate a fluorescence emission intensity from the test band in the test strip (in relative fluorescence units—RFUs—).

In some embodiments, the detectable signals in immunoassays increase over time in a linear fashion until reaching the respective maximum values for positive test samples (with the target analyte present, e.g., curves 410 and 420). However, data 451b illustrates that the signal for the negative test samples (without target analyte) increase only slowly or remain flat, or even decrease, over time. A cutoff curve 430 may be selected that separates negative test samples from a minimum detectable positive test sample, as shown in the chart. In a first test sample, no target analyte (e.g., 0 org/mL in the negative test sample) is included in the test sample and therefore the curve obtained may be used as a blank, or reference signal. In a second test sample, a mid-level concentration (e.g., 5×10³ org/mL) of the target analyte is included. In a third test sample, a high- level concentration (e.g., 8×10³ org/mL) of the target analyte is included.

As illustrates in chart 400, the resulting slopes correlate directly with the respective concentrations of target analyte in the test samples. For example, for the mid-level concentration of Strep A, the slope obtained for the fluorescence signal is about 20.6 RFUs per second (RFU/sec, cf. curve 420). For the high-level concentration of Strep A, the slope obtained is about 32.8 RFU/sec (cf. curve 410). Therefore, in some embodiments, the kinetic slopes are linear against analyte concentrations, and an analyte concentration can be reliably estimated once a function for the relationship between rate and concentration is known.

In the illustrated chart (cf. FIG. 4), it is seen that when reading an immunoassay, it is desirable to collect measurements at multiple points in time. The multiple measurements avoid the occurrence of false positives or false negatives in the assay measurements arising from significant noise at an arbitrary cutoff point. Thus, in the instance that the detector has a noise floor at up to 2×10³ RFUs, any qualitative determination of the assay result is reliably made after a time T_{A}. In some embodiments, a threshold curve 440 may be selected as a constant value above cutoff curve 430 for any foreseeable measurement time (e.g., 2×10³ RFUs). Accordingly, a qualitative determination of the assay is made when the measured signal is greater than the threshold. However, such mechanism provides a reliable determination for a mid-level analyte concentration only after a time T_{B}, which is not easily determined a-priori. Moreover, a single measurement at time T_{B} is not helpful to clearly distinguish between a mid-level target analyte concentration and a high-level target analyte concentration.

To avoid unnecessary time delays and inconsistencies in the assay results, embodiments as disclosed herein use at least two measurements (e.g., at time T_{A} and at time T_{B}) to obtain a slope value. Accordingly, the effect of noise is removed because noise contributes to a constant baseline, and by time T_{B}, a distinction with the negative test sample is established. In addition, obtaining a slope value at time T_{B} allows a distinction between the mid-level (20.6 RFUs/sec) and the high-level (32.8 RFUs/sec) target analyte test samples to be made accurately.

In some embodiments, a qualitative determination of assay results includes comparing the slopes from the negative population (e.g., 1.8 RFUs/sec) with the slopes from the positive populations (mid-level and high-level target analyte concentration, 20.6 and 32.8 RFUs/sec, respectively). A cutoff slope (e.g., 2 RFUs/sec) for the cutoff curve may be used as a pre-selected threshold. In subsequent measurements using the assay, slopes having values less than the cutoff slope (e.g., <2 RFUs/sec) are used to indicate non-binding (e.g., zero target analyte concentration) test samples. Conversely, samples for which the measured slope values are greater than the cutoff slope value indicate positive binding events. Thus the slope can itself be used to identify a positive binding event.

Use of slope measurements can therefore make assays more consistent and reliable, but without any additional significant measurement burden. There is also the advantage that false positives can be reduced or almost eliminated. In the situation that the overall signal due to the analyte is low, or similar in magnitude to background noise, a measurement of the rate will assist in separating the signal (if it exists) from background. Conversely, establishing that the rate is zero (or below the cut-off) will prevent background noise that is significant in magnitude from being incorrectly categorized as signal.

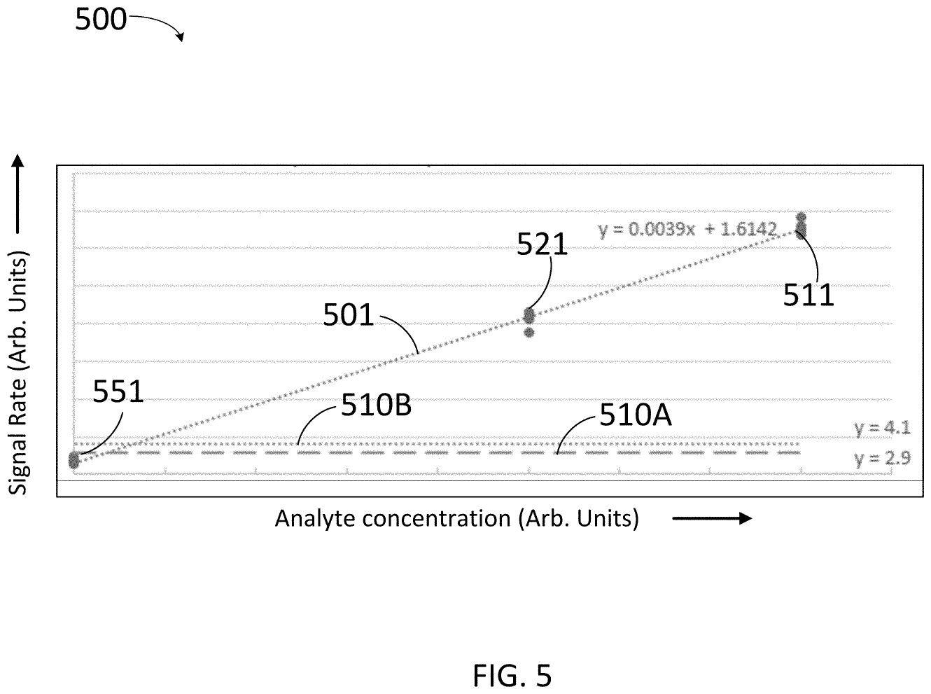

FIG. 5 is a chart 500 illustrating the slope of the curves in chart 400 as a function of analyte concentration. The abscissae in the chart indicate analyte concentration (X-Axis, arbitrary units), and the ordinates indicate the signal rate (Y-Axis, arbitrary units, e.g., the units in the ordinates in chart 400 divided by a time delta). In some embodiments, as discussed above, the units for the abscissae in the chart are org/mL, and the units for the ordinates are RFU/sec (when the signal is fluorescence emission). Data 551 in chart 500 corresponds to the negative test sample (zero analyte concentration, cf. data 451b) rendering a slope below a background 510A (e.g., signal rate 3.0 RFU/sec). Data 521 corresponds to the mid-level target analyte concentration (5×10³ org/mL) rendering a signal rate of about 20.6 RFUs/sec. And data 511 corresponds to the high-level analyte concentration (8×10⁸ org/mL) rendering a signal rate of about 32.8 RFUs/sec. Each of the multiple points in a data cluster in chart 500 (e.g., for a given analyte concentration) is associated with a single assay run from chart 400, giving a distribution of slopes (assuming that, for each assay run for chart 400, the concentration of the target analyte can be precisely controlled).

Without limitation, the data for the chart in FIG. 4 may be as follows:

TABLE I
Slopes (RFU/sec) used for the chart in FIG. 4

| Assay No. | 0 org/mL | 5 × 10³ org/mL | 8 × 10³ org/mL |
|---|---|---|---|
| 1 | 1.6033 | 18.903 | 32.983 |
| 2 | 2.4317 | 20.555 | 32.442 |
| 3 | 1.5050 | 21.025 | 34.043 |
| 4 | 1.7200 | 21.583 | 31.808 |
| 5 | 1.5050 | 20.862 | 32.692 |
| Average | 1.8 | 20.6 | 32.8 |
| SD | 0.4 | 1.0 | 0.8 |

Chart 500 enables a quantitative analysis of assay results that is simplified by the linear behavior of the kinetic slopes relative to analyte concentrations through a fit 501 of data 551. In that regard, a pre-selected threshold may be selected at background 510A or at a slightly higher value of a background 510B (e.g., 4.1 RFU/sec), to allow for a higher confidence level (albeit allowing for a slightly higher number of false negatives). In some embodiments, the sample rate levels selected as threshold in background 510A and background 510B may be obtained from the average, μ, and the standard deviation, σ, of measurements obtained in multiple assay runs (cf. last row in Table I). For example, a lower confidence level, CO, may be established for background 510A as CO=μ+3σ(=1.8+1.2=3.0 RFU/sec). In some embodiments, a higher CO may be achieved by selecting a threshold for background 510B as CO=μ+6σ(=1.8+2.4=4.2 RFU/sec). Assuming a normal distribution for all measurements, the above background 510A would render a confidence level of almost 98% of true positives above that threshold value. Likewise, the above background 510B would render a confidence level well above 99% of true positives above that threshold value.

Chart in 500 illustrates an embodiment wherein the relation between the signal rate and the analyte concentration is linear. In some embodiments, a non-linear relation can be expected, given the ranges of analyte concentration levels that may be involved. Embodiments consistent with the present disclosure may incorporate different non-linear models in the analysis of kinetic slope data from an assay. Some of the non-linear models that may be considered, without limitation, include a Michaelis-Menten kinetic model, and a “fourth party logistic” (4 PL) model, among others.

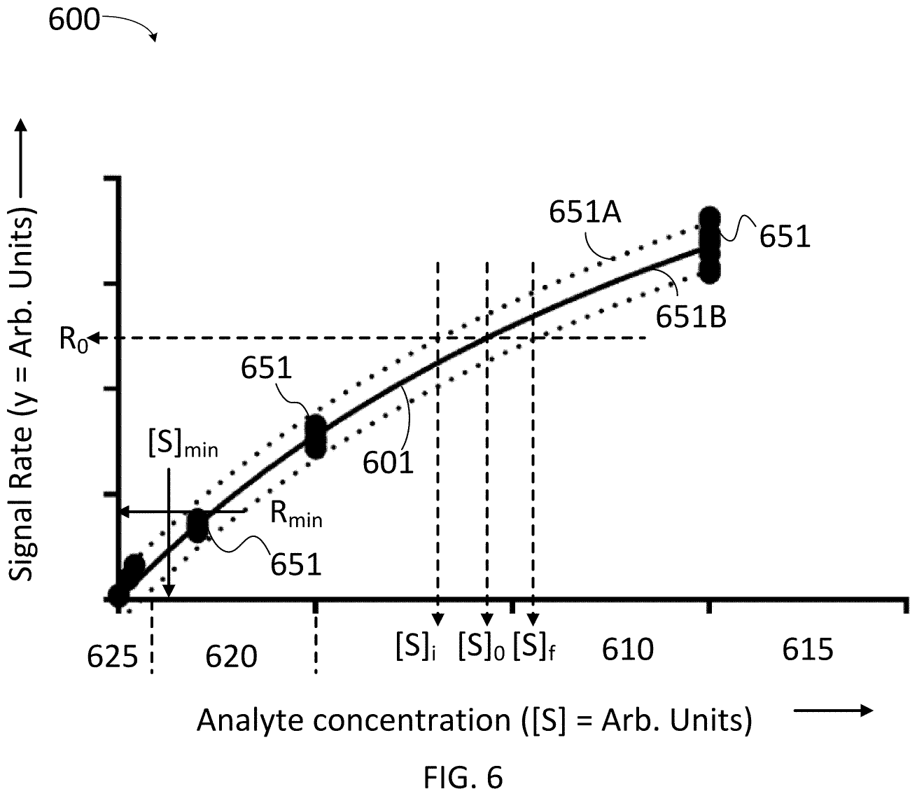

FIG. 6 is a chart 600 illustrating the slope of the curves in chart 400 (cf. chart 500 as well) as a function of analyte concentration in a Michaelis-Menten Kinetics (MMK), according to some embodiments. The ordinates and the abscissae in chart 600 are the same as those for chart 500. In an MMK model, the signal rate, R, may be expressed as a function of analyte concentration, [S], as:

$$R = \frac{{V}_{max} \cdot [ S ]}{{K}_{M} + [ S ]} \qquad ( 5.1 )$$

Where Vₘₐₓ represents the maximum rate achieved by the system (e.g., at saturating substrate concentration), and K_{M} is the Michaelis constant (e.g. the substrate concentration at which the reaction rate, R, is half of Vₘₐₓ). Note that for small values of analyte concentration [S], Eq. 5.1 predicts a linear behavior of R, much as is observed in some embodiments (cf. chart 500). Further, as the analyte concentration [S] grows, Eq. 5.1 predicts an asymptotic limit for the signal rate, R, of Vₘₐₓ. Chart 600 illustrates data 651 obtained with measurements performed for multiple assays at different analyte concentrations, including a low region 625 with linear behavior (e.g., below 10³ org/mL), and three more concentration levels: a medium-high level 620 (˜5×10⁴ org/mL), a high-level 610 (˜10⁵ org/mL), and a saturation level 615 (3×10⁵ org/mL).

A fit curve 601 indicates an accurate MMK prediction for the assay for selected values of Vₘₐₓ, and K_{M}. Further, a curve 651A is a first fiduciary that may be obtained with a set of values V^{A}ₘₐₓ, and K^{A}_{M} (cf. parameters Vₘₐₓ and K_{M}, above). Curve 651A gives a value R^{A} as in the expression:

$${R}^{A} = {δ}^{A} + \frac{{V}_{max}^{A} \cdot [ S ]}{{K}_{M}^{A} + [ S ]} \qquad ( 5.2 )$$

Where δ^{A} is an appropriately selected offset value (e.g., at zero substrate concentration [S]). Accordingly, the values V^{A}ₘₐₓ, K^{A}_{M}, and δ^{A} are selected such that for most, or all, of the sampled assays, R≤R^{A} (cf. Eq. 5.2). Likewise, a curve 651B is a second fiduciary that may be obtained with a set of values V^{B}ₘₐₓ and K^{B}M. Curve 651B gives a value RB as in the expression:

$${R}^{B} = {δ}^{B} + \frac{{V}_{max}^{B} \cdot [ S ]}{{K}_{M}^{B} + [ S ]} \qquad ( 5.3 )$$

Wherein the values V^{B}ₘₐₓ and K^{B}M, and δ^{B} (which may be a negative value) are selected such that for most, or all of the sampled assays, R^{B}≤R. Eqs. 5.1, 5.2, and 5.3 will be collectively referred to, hereinafter, as “Eqs. 5.” Hereinafter, curves 651A and 651B will be collectively referred to as “curves 651.”

In some embodiments, curves 651 may be used to establish a minimum concentration [S]ₘᵢₙ such that R^{B} is equal to zero, and a minimum rate Rₘᵢₙ=R^{A}([S]ₘᵢₙ) (cf. Eq. 5.2) is selected. Accordingly, in some embodiments, a pre-selected threshold Rₘᵢₙ is selected such that, R≤Rₘᵢₙ may be discarded as unreliable, or as zero analyte concentration results. Curves 651 may also be used to determine a confidence interval for a quantitative assessment of analyte concentration [S] given a measured signal rate value, R₀. Accordingly, a quantitative value of the concentration, [S]₀, by solving for [S] (R=R₀) in Eq. 5.1, with a confidence interval ([S]ᵢ, [S]_{f}). In some embodiments, [S]ᵢ is obtained by solving for [S](R^{A}=R₀) in Eq. 5.2 and [S]_{f} is obtained by solving for [S](R^{B}=R₀) in Eq. 5.3.

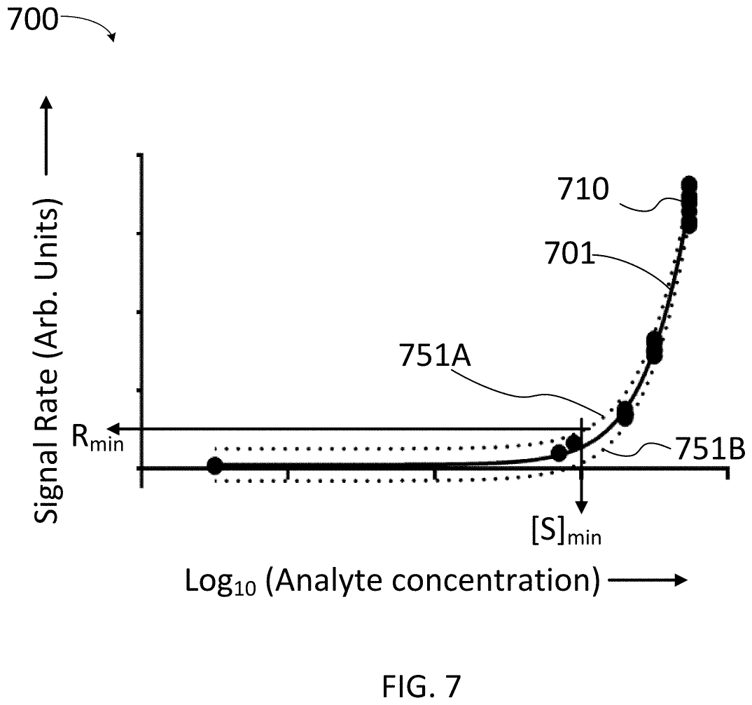

FIG. 7 is a chart 700 illustrating the slope, R, of curves 601 and 651 as a function of analyte concentration, [S], according to a 4-PL model, according to some embodiments. The ordinates in chart 700 are the same as those for the charts in FIGS. 4-5. The abscissae for chart 700 may be a logarithmic representation of the analyte concentration (e.g., a base 10 function log₁₀([S]/So)). In a 4-PL model, a fit 701 to data 710 may include a signal rate, R, expressed as a function of analyte concentration, [S], as:

$$R = d + \frac{a - d}{1 + {( \frac{[ s ]}{c} )}^{d}} \qquad ( 6.1 )$$

Wherein the parameters may be defined as: a=the minimum value that can be obtained (i.e., what happens at 0 dose); d=the maximum value that can be obtained (e.g.,. what happens at infinite dose); c=the point of inflection (i.e., the point on the S shaped curve halfway between a and d); and b=Hill's slope of the curve (e.g., the steepness of the curve at point c). In some embodiments, a<d and Eq. 6.1 renders a monotonically growing function R([S]). In some embodiments, a may be equal to zero.

Eq. 6.1 may be re-arranged to solve for [S] as a function of R (which may be measured in the assay, consistent with the present disclosure), as

$$[ S ] = {c ( \frac{a - d}{R - d} - 1 )}^{1 / b} \qquad ( 6.2 )$$

Note that for small values of analyte concentration [S], Eq. 6.1 predicts an almost constant, or slowly growing behavior of R. Further, as the analyte concentration [S] grows, Eq. 6.1 predicts a steep increase in R until [S]˜c. For values [S]>>c the curve in Eq. 6.1 tends asymptotically to the maximum value Rₘₐₓ=d. Eqs. 6.1 and 6.2 (hereinafter, collectively referred to as “Eqs. 6”) may be used for assays with a large concentration of analyte, or for which a large concentration of analyte is expected. Chart 700 illustrates measurements performed for multiple assays at different analyte concentrations, including a low region with slowly varying behavior (e.g., below 10³ org/mL), and higher concentration levels: a medium-high level (˜5×10⁴ org/mL), a high-level (˜10⁵ org/mL), and a saturation level (˜3×10⁵ org/mL). Fiduciary curves 751A and 751B (hereinafter, collectively referred to as “curves 751”) may be obtained using Eqs. 6, similarly to what was described in FIG. 5 for Eqs. 5. Likewise, curves 751 may be used similarly to the description in chart 600 to find a pre-selected threshold Rₘᵢₙ and [S]ₘᵢₙ in a 4-PL model, as disclosed herein. Also, a confidence level ([S]ᵢ, [S]_{f}) may be found in the 4-PL model using Eqs. 6 and curves 751, as described above in relation to chart 500.

In some embodiments, the analysis described in Eqs. 5 and 6, leading to charts 600 and 700 may include a multi-linear regression techniques, and a non-linear technique, such as a neural network, convolutional neural network, deep neural network, and the like. In some embodiments, Eqs. 5 and 6 may be implemented in the context of a machine learning or artificial intelligence environment, wherein a non-linear algorithm or neural network is trained with feedback from Eqs. 5 and 6 over known datasets in a supervised or unsupervised manner. In some embodiments, the non-linear algorithms may include a discriminative algorithm trained in a genetic adversarial neural network environment.

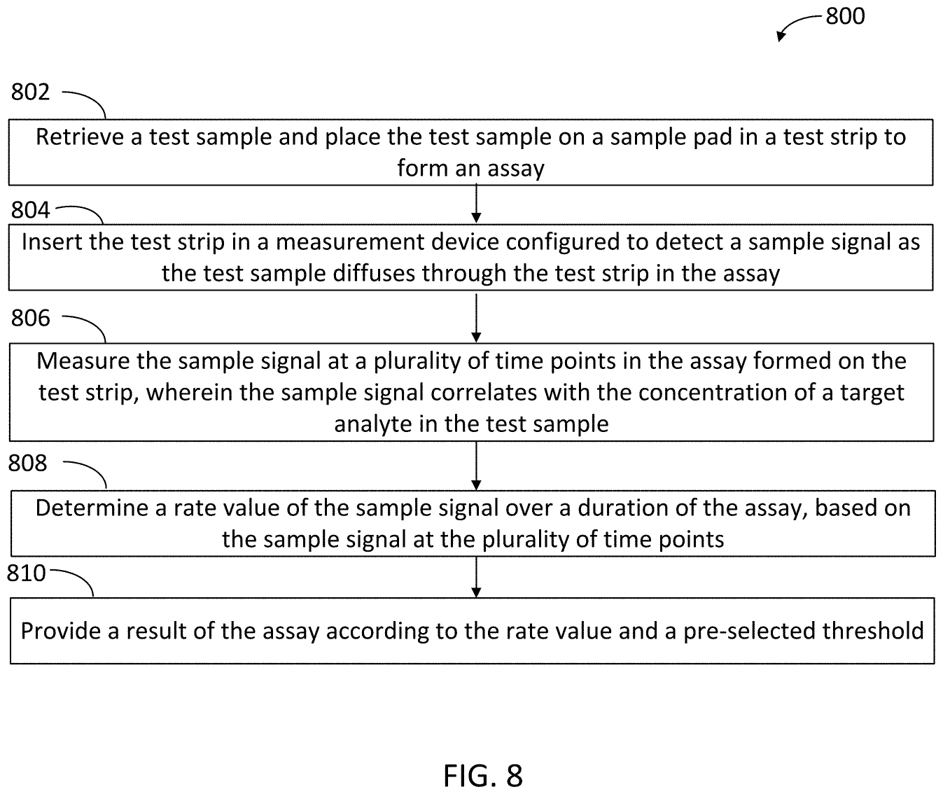

FIG. 8 is a flow chart illustrating steps in a method 800 for determining a result in an immunoassay, according to some embodiments. Method 800 may be performed at least partially by a system as in the architecture illustrated in FIG. 1. The system may include a sample receptacle configured to receive a test sample and a test strip coupled to the sample receptacle, the test strip configured to generate a signal based on a concentration of a target analyte in the test sample (e.g., system 10, sample receptacle 170, test strip 160). The system may also include a detector configured to generate a transduced signal based on the signal, and a computer configured to receive the transduced signal (e.g., detector 140, signal 151, analysis device 100, and transduced signal 141). The computer may further include a memory storing instructions, and a processor configured to execute the instructions to determine the concentration of the target analyte in the test sample (e.g., processor 112 and memory 120). The instructions include commands to perform at least some of the steps in method 800 (e.g., application 122). Methods consistent with the present disclosure may include at least one step as described in method 800. In some embodiments, methods consistent with the present disclosure include one or more steps in method 800 performed in a different order, simultaneously, almost simultaneously, or overlapping in time.

Step 802 includes retrieving the test sample and placing the test sample on a sample pad in a test strip to form an assay. In some embodiments, step 802 includes selecting, for the plurality of time points, a first time point and a last time point and the duration of the assay falls between the first time point and the last time point. In some embodiments, step 802 includes adjusting the pre-selected threshold based on a concentration of binding sites in the test strip.

Step 804 includes inserting the test strip in a measurement device configured to detect a sample signal as the test sample diffuses through the test strip in the assay.

Step 806 includes measuring the sample signal at a plurality of time points in the assay formed on the test strip, wherein the sample signal correlates with the concentration of the target analyte in the test sample.

Step 808 includes determining a rate value of the sample signal over a duration of the assay, based on the sample signal at the plurality of time points.

Step 810 includes providing a result of the assay according to the rate value and a pre-selected threshold. In some embodiments, step 810 includes comparing the rate value with the pre-selected threshold, and stopping the assay measurement when the rate value is higher than the pre-selected threshold. In some embodiments, step 810 includes allowing the assay measurement to proceed while the rate value is lesser than (or equal to) the pre-selected threshold. In some embodiments, step 810 includes running the assay on a sample free of the target analyte, measuring a signal from the test sample free of the target analyte at a second plurality of time points, obtaining a cutoff rate value based on the signal at the second plurality of time points, and determining the pre-selected threshold to be greater than the rate value of the sample signal for the sample free of the target analyte by at least 10%. In some embodiments, step 810 includes running the assay on multiple calibration samples having selected target analyte concentrations, determining multiple rate values for each of the multiple calibration samples, fitting the multiple rate values to a model based on the selected target analyte concentrations, finding a fiduciary curve based on the model, and selecting a zero of the fiduciary curve as the pre-selected threshold. In some embodiments, step 810 includes determining the pre-selected threshold according to a non-linear correlation between the rate value of the sample signal and the concentration of the target analyte in the test sample. In some embodiments, step 810 includes determining the pre-selected threshold, according to a confidence level that the concentration of the target analyte in the test sample is zero. In some embodiments, step 810 includes transmitting the result of the assay to a remote server. In some embodiments, step 810 includes determining the pre-selected threshold, according to a non-linear model correlating the concentration of the target analyte with the rate value of the sample signal.

FIG. 9 is a flow chart illustrating steps in a method 900 for determining an endpoint in an assay with a remote server, according to some embodiments. Method 900 may be performed at least partially by a system as in the architecture illustrated in FIG. 1. The system may include a sample receptacle configured to receive a test sample and a test strip coupled to the sample receptacle, the test strip configured to generate a signal based on a concentration of a target analyte in the test sample (e.g., system 10, sample receptacle 170, test strip 160). The system may also include a detector configured to generate a transduced signal based on the signal, and a computer configured to receive the transduced signal (e.g., detector 140, signal 151, analysis device 100, and transduced signal 141). The computer may further include a memory storing instructions, and a processor configured to execute the instructions to determine the concentration of the target analyte in the test sample (e.g., processor 112 and memory 120). The instructions include commands to perform at least some of the steps in method 900 (e.g., application 122). Methods consistent with the present disclosure may include at least one step as described in method 900. In some embodiments, methods consistent with the present disclosure include one or more steps in method 900 performed in a different order, simultaneously, almost simultaneously, or overlapping in time.

Step 902 includes collecting, with a sensor array, a sample signal from a test sample at a plurality of time points in an assay formed on a test strip, wherein the sample signal correlates with a concentration of a target analyte in the test sample.

Step 904 includes providing a transduced signal from the sensor array to a remote server for determining the endpoint in the assay and a result of the assay. In some embodiments, step 904 includes buffering the transduced signal over a period of time encompassing the time points, and providing a time sequence of the transduced signal to the remote server after the period of time.

Step 906 includes receiving, from the remote server, the result of the assay when the endpoint is reached. In some embodiments, step 906 includes receiving, from the remote server, an error message based on a quality of the transduced signal. In some embodiments, step 906 includes adjusting the position of the test sample in a testing device in response to an error message from the remote server. In some embodiments, step 906 includes receiving an error message when the remote server fails to find the endpoint in the assay. In some embodiments, step 906 includes receiving a confidence level for the result from the assay. In some embodiments, step 906 includes receiving, from the remote server, an expected time for reaching the endpoint in the assay. In some embodiments, step 906 includes receiving, from the remote server, an error message, and collecting a new sample signal from a new test sample in response to a request from the remote server.

Step 908 includes displaying, for a user, the result of the assay. In some embodiments, step 908 includes clearing the test strip from a testing device when the endpoint is reached and the result of the assay is negative. In some embodiments, step 908 includes displaying a rate value of the sample signal over a duration of the assay.

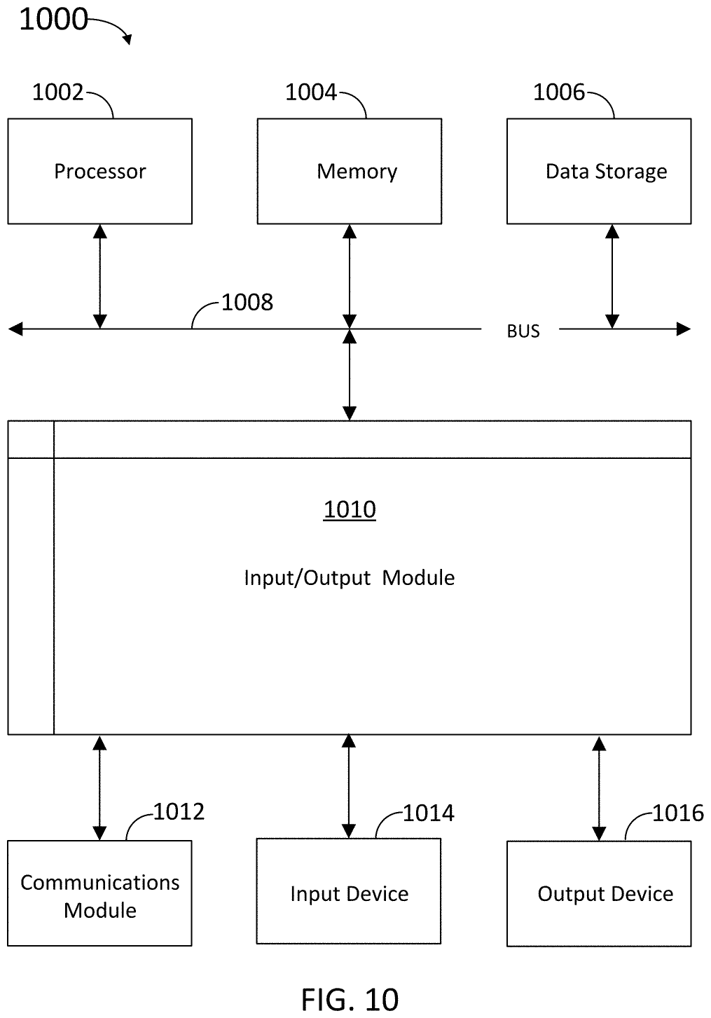

FIG. 10 is a block diagram illustrating an example computer station 1000 with which the system of FIG. 1, and the methods as disclosed herein can be implemented, according to some embodiments. In certain aspects, computer station 1000 may be implemented using hardware or a combination of software and hardware, either in a dedicated server, or integrated into another entity, or distributed across multiple entities. Computer station 1000 includes a bus 1008 or other communication mechanism for communicating information, and a processor 1002 coupled with bus 1008 for processing information. By way of example, computer station 1000 may be implemented with one or more processors 1002. Processor 1002 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer station can include, in addition to hardware, code that creates an execution environment for the computer program in question, e.g., code that constitutes processor firmware, a protocol stack, a database management system, an operating system, or a combination of one or more of them stored in an included memory 1004, such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled to the bus for storing information and instructions to be executed by the processor. Processor 1002 and memory 1004 can be supplemented by, or incorporated in, special purpose logic circuitry.

The instructions may be stored in memory 1004 and implemented in one or more computer program products, i.e., one or more modules of computer program instructions encoded on a computer-readable medium for execution by, or to control the operation of, computer station 1000, and according to any method well-known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command-line interface languages, compiled languages, concurrent languages, curly-bracket languages, dataflow languages, data-structured languages, declarative languages, esoteric languages, extension languages, fourth-generation languages, functional languages, interactive-mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 1004 may also be used for storing temporary variable or other intermediate information during execution of instructions to be executed by processor 1002.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors 1002 executing one or more computer programs to perform functions by operating on input data and generating output.

Computer station 1000 further includes a data storage device 1006 such as a magnetic disk or optical disk, coupled to the bus for storing information and instructions. Computer station 1000 may be coupled via an input/output module 1010 to various devices. Input/output module 1010 can be any input/output module. Exemplary input/output modules 1010 include data ports such as USB ports. Input/output module 1010 is configured to connect to a communications module 1012. Exemplary communications modules 1012 include networking interface cards, such as Ethernet cards and modems. In certain aspects, input/output module 1010 may be configured to connect to a plurality of devices, such as an input device 1014 and/or an output device 1016. Exemplary input devices 1014 include a keyboard and a pointing device, e.g., a mouse or a trackball, by which a user can provide input to the computer station. Other kinds of input devices 1014 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Exemplary output devices include display devices, such as an LCD (liquid crystal display) monitor for displaying information to the user.

In some embodiments, computer station 1000 is a network-based, voice-activated device accessed by the user. The input/output device may include a microphone providing the queries in voice format, and receiving multiple inputs from the user also in a voice format, in the language of the user. Further, in some embodiments, a neural linguistic algorithm may cause the voice-activated device to contact the user back and receive a user selection of the respiratory mask via a voice command or request.

According to one aspect of the present disclosure, an image-capturing device and server can be implemented using computer station 1000 in response to processor 1002 executing one or more sequences of one or more instructions contained in memory 1004. Such instructions may be read into memory 1004 from another machine-readable medium, such as the data storage device 1006. Execution of the sequences of instructions contained in the main memory 1004 causes the processor to perform the process steps described herein. One or more processors in a multi-processing arrangement may also be employed to execute the sequences of instructions contained in the memory. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back-end component, e.g., as a data server, or that includes a middleware component, e.g., an application server, or that includes a front-end component, e.g., an image-capturing device having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. The communication network can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer station 1000 can include image-capturing devices and servers wherein the image-capturing device and server are generally remote from each other and typically interact through a communication network. The relationship of image-capturing device and server arises by virtue of computer programs running on the respective computers and having an image-capturing device-server relationship to each other. Computer station 1000 can be, for example, and without limitation, a desktop computer, laptop computer, or tablet computer. Computer station 1000 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to the processor for execution. Such a medium may take many forms, including, but not limited to, non-volatile media, volatile media, and transmission media. Non-volatile media include, for example, optical or magnetic disks, such as data storage device 1006. Volatile media include dynamic memory, such as the memory. Transmission media include coaxial cables, copper wire, and fiber optics, including the wires that comprise the bus. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

To the extent that the term “include,” “have,” or the like is used in the description or the claims, such term is intended to be inclusive in a manner similar to the term “comprise” as “comprise” is interpreted when employed as a transitional word in a claim. The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

In one aspect, a method may be an operation, an instruction, or a function and vice versa. In one aspect, a claim may be amended to include some or all of the words (e.g., instructions, operations, functions, or components) recited in other one or more claims, one or more words, one or more sentences, one or more phrases, one or more paragraphs, and/or one or more claims.

The foregoing description is intended to illustrate various aspects of the instant technology. It is not intended that the examples presented herein limit the scope of the appended claims. The invention now being fully described, it will be apparent to one of ordinary skill in the art that many changes and modifications can be made thereto without departing from the spirit or scope of the appended claims.

To illustrate the interchangeability of hardware and software, items such as the various illustrative blocks, modules, components, methods, operations, instructions, and algorithms have been described generally in terms of their functionality. Whether such functionality is implemented as hardware, software, or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

While this specification contains many specifics, these should not be construed as limitations on the scope of what may be described, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially described as such, one or more features from a described combination can in some cases be excised from the combination, and the described combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the described subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately described subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

It is claimed:

1. A method of determining an endpoint in an assay, the method comprising:
measuring a sample signal from a test sample at a plurality of time points in an assay formed on a test strip, wherein the sample signal correlates with a concentration of a target analyte in the test sample;
determining a rate value of the sample signal over a duration of the assay based on the sample signal at the plurality of time points; and
providing a result of the assay according to the rate value and a pre-selected threshold.

2. The method of claim 1, further comprising selecting, for the plurality of time points, a first time point and a last time point and the duration of the assay falls between the first time point and the last time point.

3. The method of claim 1, further comprising adjusting the pre-selected threshold based on a concentration of binding sites in the test strip.

4. The method of any one of claim 1, further comprising:
running the assay on a sample free of the target analyte;
measuring a signal from the test sample free of the target analyte at a second plurality of time points;
obtaining a cutoff rate value based on the signal at the second plurality of time points; and
determining the pre-selected threshold to be greater than the rate value of the sample signal for the sample free of the target analyte by at least 10%.

5. The method of any one of claim 1, further comprising:
running the assay on multiple calibration samples having selected target analyte concentrations;
determining multiple rate values for each of the multiple calibration samples;
fitting the multiple rate values to a model based on the selected target analyte concentrations; finding a fiduciary curve based on the model; and
selecting a zero of the fiduciary curve as the pre-selected threshold.

6. The method of any one of claim 1, further comprising determining the pre-selected threshold according to a non-linear correlation between the rate value of the sample signal and the concentration of the target analyte in the test sample.

7. The method of any one of claim 1, further comprising determining the pre-selected threshold according to a confidence level that the concentration of the target analyte in the test sample is zero.

8. The method of any one of claim 1, further comprising determining the pre-selected threshold according to a non-linear model correlating the concentration of the target analyte with the rate value of the sample signal.

9. The method of any one of claim 1, further comprising determining a first time point in the plurality of time points when an expected rate value of the sample signal is different from zero.

10. The method of any one of claim 1, further comprising transmitting the result of the assay to a remote server.

11. A system, comprising:
a sample receptacle configured to receive a test sample;
a test strip coupled to the sample receptacle, the test strip configured to generate a signal based on a concentration of a target analyte in the test sample;
a detector configured to generate a transduced signal based on the signal;
and a computer configured to receive the transduced signal, the computer further comprising:
a memory storing instructions; and
a processor configured to execute the instructions to determine the concentration of the target analyte in the test sample, wherein the instructions include commands to:
retrieve the transduced signal from the detector at multiple time points,
determine a signal rate based on a signal value for at least two of the time points, and
determine the concentration of the target analyte based on the signal rate and a model.

12. The system of claim 11, wherein the test strip comprises a label pad, and a test band, wherein the label pad comprises a concentration of multiple label complexes configured to attach to the target analyte in the test sample and diffuse with the test sample along the test strip toward the test band, and the test band comprises an immunoassay configured to bind the target analyte with at least one of the label complexes to a substrate, and wherein the label complexes are further configured to generate the signal.

13. The system of claim 11, wherein the test strip comprises a control band configured to provide a blank signal for the detector, and wherein the computer is configured to use the blank signal as a background to determine the concentration of the target analyte in the test sample.

14. The system of claim 11, wherein the memory stores a pre-selected threshold and instructions which, when executed by the processor, cause the system to provide the concentration of the target analyte when the signal rate exceeds the pre-selected threshold.

15. The system of claim 11, wherein the memory stores instructions which, when executed by the processor, cause the system to fit at least one parameter in the model to multiple values of the transduced signal obtained at multiple time intervals.

16. The system of claim 11, wherein the computer further comprises a communications module configured to transmit the concentration of the target analyte to a remote server.

17. The system of claim 11, wherein the test strip further comprises multiple channels, each of the multiple channels comprising at least one control dot providing a control signal to separately determine the concentration of the target analyte in the sample.

18. The system of claim 11, wherein the test strip further comprises multiple channels, each of the multiple channels comprising at least one control dot providing a control signal to jointly determine the concentration of the target analyte in the sample.

19. The system of claim 11, further comprising a communications module configured to provide the transduced signal to a remote server, and to receive a result from the remote server, the result indicative of a presence or an absence of a disease in a patient.

20. The system of claim 11, wherein the detector is a detector array and the transduced signal is an image of the test strip.
